(19) United States
(12) Plant Patent Application Publication
(10) Pub. No.: US 2020/0205329 P1
(43) Pub. Date: Jun. 25, 2020

(54) Grape plant named 'VOLTURNIS'

(50) ;

(71) Applicants: UNIVERSITÀ DEGLI STUDI DI UDINE, Udine (IT); ISTITUTO DI GENOMICA APPLICATA, Udine (IT)

(72) Inventors: Simone Diego Castellarin, Vancouver (CA); Guido Cipriani, Faedis (IT); Gabriele Di Gaspero, Cividale del Friuli (IT); Michele Morgante, Tricesimo (IT); Enrico Peterlunger, Codroipo (IT); Raffaele Testolin, Udine (IT)

(73) Assignees: UNIVERSITÀ DEGLI STUDI DI UDINE, Udine (IT); ISTITUTO DI GENOMICA APPLICATA, Udine (IT)

(21) Appl. No.: 16/602,628

(22) Filed: Nov. 12, 2019

(30) Foreign Application Priority Data
Dec. 20, 2018 (EU) 20183517

(51) Int. Cl. A01H 6/88

(52) U.S. Cl. PLT/205

(57) ABSTRACT

A new grape variety distinguished by its high vigour with a semi-erect growth habit, low-medium blistering of the upper side of the leaf blade, circular shaped leaves, fruit clusters of medium or medium-large bunch density with 3-4 wings, medium harvesting time (early September in northeastern Italy), and resistance to downy mildew.

### CROSS REFERENCE TO RELATED APPLICATIONS

This application claims the benefit of priority from EU Community Plant Variety Rights (CPVR) Application No. 20183517, filed Dec. 20, 2018.

Latin name of the genus and species of the plant claimed: Vitis vinifera L.

Variety denomination: ‘VOLTURNIS’.

### BACKGROUND

The present application relates to a new and distinct variety of grape named ‘VOLTURNIS’. The new plant resulted from a planned hybridization program and is a selection from crossing 99-1-48 (unpatented) as the seed parent with Pinot Noir (unpatented) as the pollen parent in 2007. The resulting plant was selected in 2013 when growing in a cultivated area in Udine, Italy.

### SUMMARY

The ‘VOLTURNIS’ variety is distinguished from other grape varieties due to the following unique combination of characteristics: high vigour with semi-erect growth habit, low-medium blistering of upper side of leaf blade, circular shaped leaves, fruit cluster of medium or medium-large bunch density with 3-4 wings, medium harvesting time (early September in northeastern Italy), and resistance to downy mildew.

| Character- | | Male Parent |
|---|---|---|
| istic | ‘VOLTURNIS’ | Pinot noir |
| vigour | High | Medium |
| growth habit | Semi-erect | Horizontal |
| leaf | Medium, medium to dark | Medium, dark green color |
| | green color (upper surface) | (upper surface) N134A, |
| | N134A, low-medium | medium blistering of upper |
| | blistering of upper side of | side of blade pentagonal |
| | blade, circular shape, small | shape, small-medium teeth |
| | teeth | |
| cluster | Medium or medium-large | Low weight, with one or |
| | conical clusters, tendentially | two wings, compact, broad |
| | compact with 3-4 wings, berry | ellipsoid berry, berry skin |
| | is medium-to-small and | with blue-black color 99A, |
| | globose, blue-black coloured | soft flesh, neutral taste, no |
| | 99A rather thick skin with | flesh coloration |
| | thick pruinosity, soft pulp | |
| | with neutral flavour | |
| harvesting | Medium [early September | Early [end of August |
| time | (Middle Friuli, northeastern | (Middle Friuli, |
| | Italy)] | northeastern Italy)] |
| resistances | Resistant to downy mildew, | No resistance to downy |
| | no resistance to powdery | mildew, no resistance to |
| | mildew | powdery mildew |

Asexual reproduction of this new variety by vegetative cuttings shows that the foregoing characteristics come true to form, are firmly fixed, and are established and transmitted through succeeding propagations.

Certain characteristics of this variety may change with changing environmental conditions (such as photoperiod, temperature, moisture, soil conditions, nutrient availability, or other factors). Color descriptions and other terminology are used in accordance with their ordinary dictionary descriptions, unless the context clearly indicates otherwise. Color designations (hue/value/chroma) are made with reference to The Royal Horticultural Society (R.H.S.) Colour Chart, 5^{th} edition, London, England, 2007.

### BRIEF DESCRIPTION OF THE DRAWINGS

FIG. 1 is a photograph showing a shoot of the new variety.

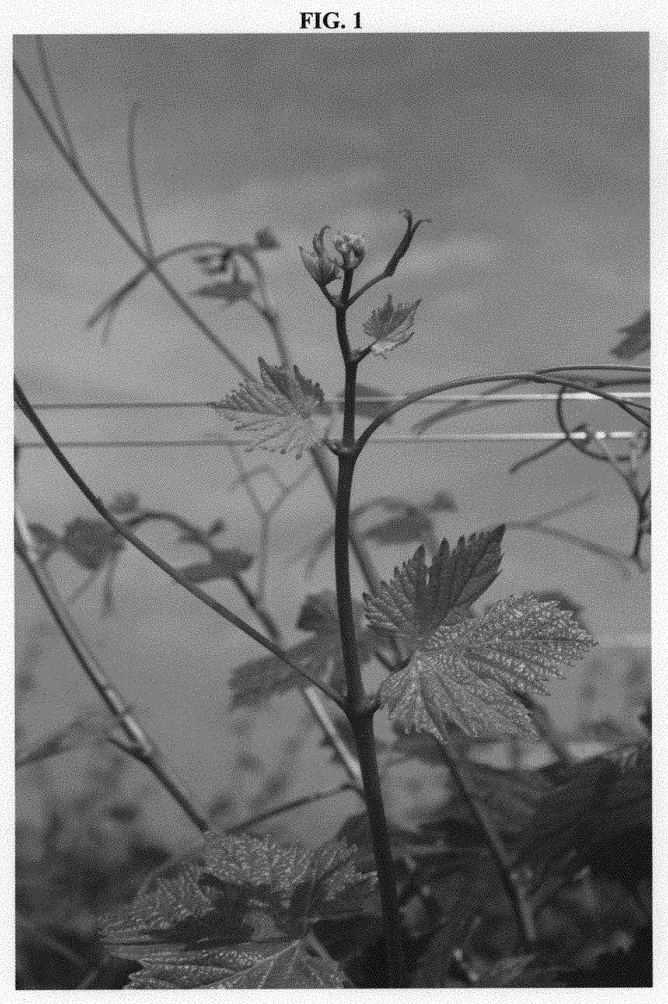

FIG. 2 is a photograph showing a leaf of the new variety.

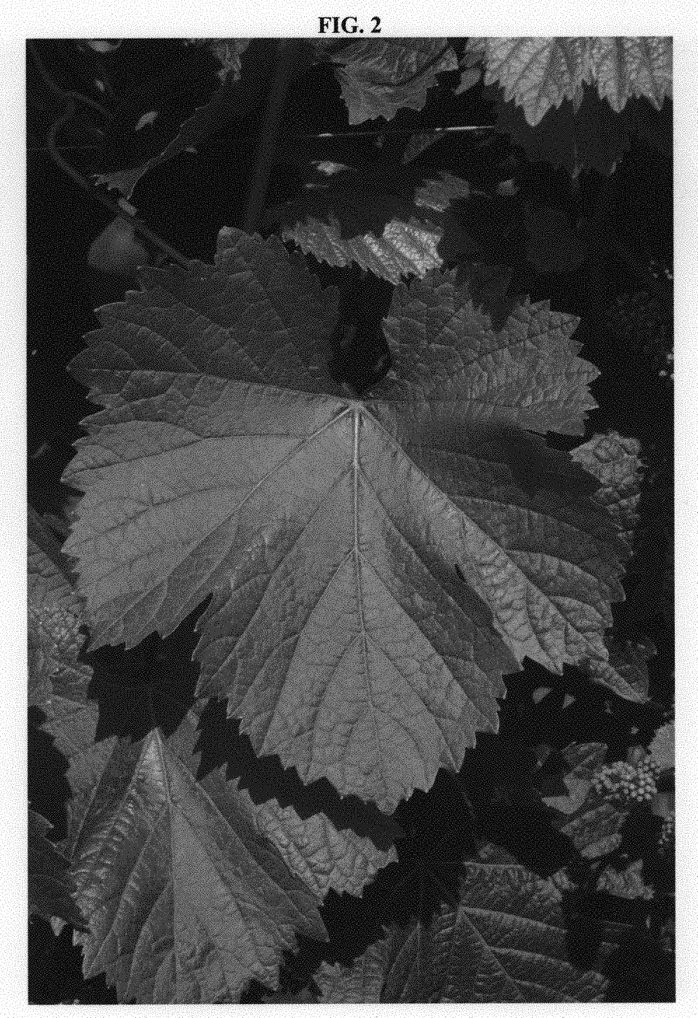

FIG. 3 is a photograph showing fruit of the new variety.

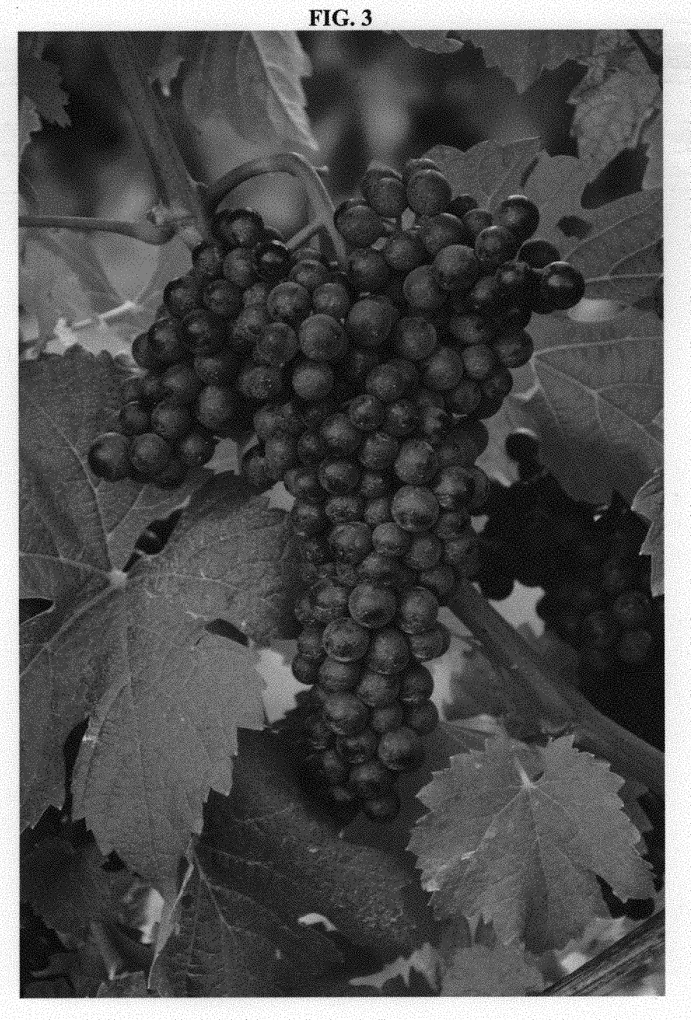

FIG. 4 is a photograph showing plants of the new variety.

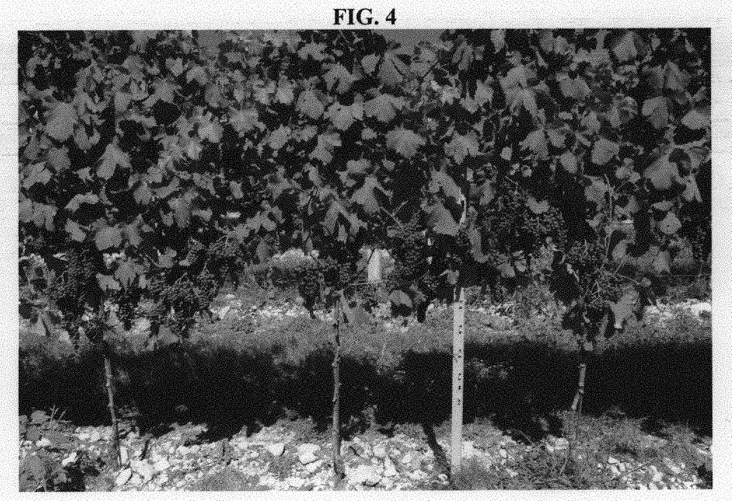

The color photographs shows typical specimens of this new variety and depict the color as nearly true as is reasonably possible to make the same in a color illustration of this character. It should be noted that colors may vary, for example due to lighting conditions at the time the photograph is taken. Therefore, color characteristics of this new variety should be determined with reference to the observations described herein, rather than from the photograph alone.

### DETAILED DESCRIPTION

The new variety was identified due to its resistance to downy mildew and powdery mildew, and was suited to the climatic and soil conditions of central-northern Italy. The new variety can be used in wine production.

### BOTANICAL

The following detailed description of the ‘VOLTURNIS’ variety is based on observations of asexually reproduced progeny. The observed progeny are plants which were 3-5 years of age. The following detailed description concerns the plants growing in an open field taken in Rauscedo, Italy in 2016-2018. The original plant and progeny have been observed growing in a cultivated area in Rauscedo, Italy, with medium texture soil that is rich in skeleton and alluvial in nature. Temperatures in Rauscedo, Italy range from a high of 29° C. to a low of −2° C. Average rainfall is 822 mm per year, with an average rainfall during the growing season (April-September) of 453 mm.

The chart used in the identification of colors described herein is The R.H.S. Colour Chart, 5^{th} edition, except where general color terms of ordinary significance are used. The color values were determined in August-September 2018 under natural light conditions in Rauscedo, Italy.
Scientific name: Vitis vinifera L.
Parentage:
   Seed parent.—99-1-48.
   Pollen parent.—Pinot Noir.
Plant:
   Vigor.—High.
   Growth habit.—Semi-erect.
Shoot:
   Opening of the shoot tip.—Fully open for young shoot.
   Distribution of the anthocyanin coloration of the prostrate hairs of the shoot tip.—Piping.
   Density of prostate hair on the shoot tip.—Low.
   Attitude (before tying).—Semi-erect.
   Color of the dorsal side of internodes.—Green 140B.
   Color of the ventral side of internodes.—Green 140B.
   Distribution of anthocyanin coloration on the bud scales.—Absent.
   Number of consecutive tendrils.—2 or less.
Leaves:
   Shape.—Circular.
   Teeth.—Small, mixture between both sides straight and both sides convex.
   Blistering.—Low-medium blistering of upper side of blade.
   Size.—Medium.
   Young leaf.—Color of upper surface is green 134B, with sparse prostrate hairs between main veins on the lower side of blade.
   Mature leaf.—Color of upper surface is medium-to-dark green N134A, with sparse prostrate hairs between main veins on the lower side of blade.
   Mature leaf.—Five lobes.
   Area of anthocyanin coloration of main veins on upper side and lower of mature blade.—Only at the petiolar point.
   Area of anthocyanin coloration of main veins on u lower of mature blade.—Absent.
   Goffering of mature blade.—Absent or very weak.
   Undulation of blade between main or lateral veins of mature leaf.—Absent.
   Degree of opening.—Open.
   Shape of base of petiole sinus.—Brace-shaped.
   Petiole sinus base limited by vein.—Not limited.
   Density of prostrate hairs on petiole.—None or very low.
   Density of erect hairs on petiole.—None or very low.
   Depth of upper lateral sinuses.—Medium.
Flowers: Fully developed stamens and fully developed gynoecium
   Insertion of 1st inflorescence.—Up to the 2^{nd} node.
   Number of inflorescences per shoot.—1 to 3.
Fruit:
   Cluster.—Medium or medium-large conical clusters, tendentially compact with 3-4 wings.
   Time of beginning of berry ripening.—Early.
   Berry shape.—Globose.
   Berry size.—Medium-to-small.
   Pruinosity.—Thick.
   Pulp.—Soft.
   Color of skin (without bloom).—Blue black 99A.
   Skin.—Medium-to-thick.
   Anthocyanin coloration of flesh.—Absent or very weak.
   Flavor.—None/neutral.
   Formation of seeds.—Complete.
   Harvest time.—Medium (early September in northeastern Italy).
   Bunch length (peduncle excluded).—Medium.
   Bunch width.—Medium-wide.
   Bunch density.—Medium-to dense.
   Berry hilium.—Apparent.
Must characteristics:
   Sugars (Brix).—22.
   pH.—3.6.
   Total acidity.—6.6 g/l.
   Tartaric acid.—8.39 g/l.
   Malic acid.—3.31 g/l.
Production characteristics:
   Clusters per shoot.—1.68.
   Grape production.—3600 g/plant.
   No. of bunches/vine (at harvest).—19.4.
   Average weight of the bunch.—263.3 g.
   Average berry weight.—1.43 g.
   Pruning wood weight.—730 g/plant.
   Index of ravaz.—4.93.
Wine produced from grapes:
   Total acidity.—5.3 g/l.
   Tartaric acid.—1.77 g/l.
   pH.—3.83.
   Net extract.—29.6 g/l.
   Flavonoids.—1486 mg/l.
   Anthocyanins.—351 mg/l.
   Total polyphenols.—1807 mg/l.
   Alcohol.—14.1 g/l.
   Volatile acidity.—0.7 g/l.
   Reducing sugars.—1.4 g/l.
Molecular analysis:

| VVS2 | | VVMD5 | | VVMD7 | | VVMD25 | | VVMD27 | |
|---|---|---|---|---|---|---|---|---|---|
| N + 10 | N + 14 | N + 6 | N + 16 | N + 8 | N + 22 | N + 4 | N + 14 | N + 4 | N + 10 |

| VVMD28 | | VVMD32 | | VRZAG 62 | | VRZAG 79 | |
|---|---|---|---|---|---|---|---|
| N + 2 | N + 20 | N + 37 | N + 39 | N + 20 | N + 26 | N + 2 | N + 14 |

Use international coding based on “N” (see European project GENRES 081—A basis for the preservation and utilization of Vitis genetic resources)
Phenological characteristics (in Rauscedo, Italy):
   Germination.—March 31 4.
   Flowering.—June 2.
   Veraison (change of color).—August 2.
   Maturation.—September 17.
Use: Wine grape.
Disease/pest resistance: Resistance to downy mildew, but no resistance to powdery mildew.

We claim:

1. A new and distinct variety of Vitis vinifera L. plant substantially as illustrated and described herein.
